(19) United States
(12) Patent Application Publication
(10) Pub. No.: US 2011/0275133 A1
(43) Pub. Date: Nov. 10, 2011

(54) Proteinaceous Digestates

(75) Inventor: John William Carson, Burton-on-Trent (GB)

(73) Assignee: TANKS AND VESSELS INDUSTRIES LIMITED, Doncaster, South Yorkshire (GB)

(21) Appl. No.: 13/128,962

(22) PCT Filed: Nov. 11, 2009
(86) PCT No.: PCT/GB09/51512

§ 371 Date: Jul. 25, 2011

(30) Foreign Application Priority Data
Nov. 14, 2008 (GB) 0820846.4

(51) Int. Cl. C12S 3/14; C12N 9/00

(52) U.S. Cl. 435/183; 435/272

(57) ABSTRACT

A method of aiding the digestion of a proteinaceous material, the method comprising adding to a sample of proteinaceous raw material a composition comprising fruit, fruit waste or an enzyme derived therefrom. A digestate liquor obtained by said method is also described.

The present invention relates to a process by which proteinaceous digestates can be manufactured from proteinaceous materials. The process of the present invention and the product obtained provide significant advantages compared with processes and products of the prior art. Examples of such advantages are discussed herein.

According to a first aspect of the present invention there is provided a method of aiding the digestion of a proteinaceous material, the method comprising adding to a sample of proteinaceous raw material a composition comprising fruit, fruit waste or an enzyme derived therefrom.

The method of the present invention is preferably a method of improving the digestion of a proteinaceous material. The method may accelerate the digestion of the proteinaceous material. In some cases the method may provide a digestate liquor having improved properties and/or which has been digested to a greater extent, compared to an equivalent digestate liquor obtained by a similar process in which the composition comprising fruit, fruit waste or an enzyme derived therefrom is not added.

According to a second aspect of the present invention there is provided a product obtained by the process of the first aspect. This product may be known as a digestate liquor.

Preferred features of the process of the present invention and the product obtained thereby are further defined herein.

The present invention may suitably provide a process for the manufacture of shorter chain poly-peptides, that is oligomeric proteins, and proteinaceous products with modified end groups and amino acids. Such protein-derived entities may be used in agronomic nutrification, animal feedstuffs, speciality human foods, bio-chemical intermediates and pharmaceutical compositions.

The process of the present invention suitably involves two inter-related reactions: microbial digestion and enzymic digestion. This inter-related, that is dual process, produces an intermediate product, namely a digestate liquor.

It is a preferred feature of the present invention that the combination of microbial and enzymic digestion processes produces different, that is advantageous, digestates than the known techniques of either aerobic or anaerobic microbial digestion.

Moreover, the present invention desirably provides a faster manufacturing route, with significantly reduced fuel cost, for the production of advantageous microbial enzymic digestates.

The present invention may be described as a Co-enzymically accelerated Aerobic Microbial digestion approach to Endogenous Organics; (CAMEO).

The intermediate product produced by the process defined herein, that is the digestate liquor, may be used without further processing in such compositions as organic fertilisers and animal feeds. Alternatively, the immediate product may be further processed, by a variety of chemical and bio-chemical separation and extraction techniques to yield oligomeric proteins and modified peptides of value as products in the fields of speciality human foods, bio-chemical intermediates and pharmaceutical compositions.

Our research and development is continuing into these separation and extractive techniques and into applications for the specific products that can be obtained from the digestate liquor. The present invention suitably relates to the process by which the improved microbial enzymic digestate is manufactured and the composition of matter of the microbial enzymic digestate produced.

Whereas it is known that by the application of techniques such as aerobic and anerobic digestion, amino acids and partially digested protein fragments can be produced, the products of the prior art in the form of digestates are of low commercial value. The present inventors have found that by introducing co-enzymic entities, to the digestion process, the digestates produced suitably have higher value oligomeric and modified oligomeric compositions. It is believed that this effect is produced via the presence of these co-enzymes which work synergistically with microbial enzymes, to accelerate and improve the digestion process. Preferably this synergistic reaction quickly produces a fully pasteurised digestate without the need for auxiliary heating, and contained within the digestate are shorter chain length proteinaceous oligomers of high value in the end uses envisaged.

It is well known that microbial digestion techniques depend to a greater or lesser extent on the generation and release of enzymic reagents by the micro-organisms concerned. Specifically, it is known that aerobic microbes exude a range of enzymic reagents of the protease type. These, in an oxidising environment digest proteinaceous materials via oxidative cleavage of the peptide bonds in the protein molecule.

It is also known that various enzymic reactions can be catalysed by the presence of co-enzymes.

The present inventors have found that when certain fruit wastes were added to an aerobic digester containing proteinaceous wastes, the effect was an acceleration of the digestion process accompanied by the evolution of significant heat of reaction. The result was a fully pasteurised extensively de-polymerised digestate, involving a reaction time significantly reduced in comparison to previous experiments.

Experimentation within specific fruits showed that the reaction acceleration effect achieved was enhanced by the presence in the fruit waste of specific co-enzymic entities. The inventors found that, while most fruits have some positive effect on the reaction, certain fruits gave a greater acceleration effect than others. Specifically the papaya group of fruits gave the maximum acceleration. It is belived that this effect is due to the presence, in the fruit, of the specific enzymic entity known as papease.

Thus in preferred embodiments the composition added to the proteinaceous material in the method of the first aspect of the present invention comprises fruit, fruit waste or an enzyme derived from papaya or the papaya group of fruit. Preferably the composition comprises papease.

The present inventors believe that fruit-born enzymic entities can act as co-enzymic accelerants with microbially generated protease, when present in aerobic digestion reactions.

Moreover, our on-going research has shown that by careful control of the oxidative environment within aerobic digesters, this co-enzymic acceleration effect can be optimised. Reactor pH should also be controlled to maximise the thoroughness of digestion.

The specific development of microbial digestion which is the subject of the present invention will now be defined.

The present invention is suitably an adaptation of aerobic digestion rather than anaerobic digestion.

Preferably the adaptation of conventional aerobic digestion, that is the subject of the present invention, comprises the introduction of co-enzymic entities and the control of the oxidising potential and the pH of the digestion reaction as it proceeds to completion.

The present inventors have found that aerobic digestion can be controlled such that the environment involved in the microbial digestion process is either strongly oxidative, that is oxygen-rich or weakly oxidative, that is approaching anaerobic conditions. By controlling the oxidative potential of the atmosphere within the digester, modifications can be made to both the mode of digestion and the products of digestion contained in the digestate. This controlled effect stems from the combination of microbial, enzymic and co-enzymic oxidation reactions taking place. Preferably the pH of the reaction is also controlled.

The inventors have found that the reduction in overall time-span involved in an aerobic microbial digestion reaction can be maximised, that is the reactor dwell time can be minimised, via the addition of fruit derived co-enzymes of the papease type, and the control of reactor aeration, temperature, and pH during the digestion process.

To maximise this acceleration effect it is advantageous to reduce aeration in the early stages of the process and then progressively increase aeration as the process progresses.

Without wishing to be bound by the theory, it is believed that for the effectiveness of the above aeration regime is may be explained as follows: in the early stages there are relatively few micro-organisms and therefore excess aeration only cools the reaction. As the population of micro-organisms grows more aeration is required for respiration. The increased population produces more enzymes and these are catalysed by the presence of the added co-enzymes. Then a point is reached at which the reaction temperature pasteurises the micro-organism population. However, further aeration at that stage accelerates the enzymic reaction. This enzymic oxidation reaction is exothermic and exceptionally high temperatures are achieved. The result can be a digestate liquor with maximised protein digestion, at an end point temperature close to 100° C.

Preferably the present invention provides a method a producing a digestate liquor, the method comprising:
(a) adding to an aerobic digestor reactor a composition comprising a proteinaceous material and a composition comprising fruit, fruit waste or an enzyme derived therefrom; and
(b) allowing the resultant mixture to remain in the reactor for a period sufficient for digestion of the proteinaceous material to occur.

Preferably, step (b) comprises allowing the resultant mixture to remain for a period sufficient to allow substantially all of the proteinaceous material to be digested to at least some extent.

During step (b) the aeration in the reactor may be varied.

Suitably during step (b) the temperature in the reactor is allowed to increase.

Preferably during step (b) the pH within the reactor is maintained between pH5 and pH8.

In some embodiments a pH control agent is added to the reactor in step (a).

Preferred features of the process by which accelerated co-enzymic microbial digestion is achieved, will now be further described:

Suitably, the first step is to charge the aerobic reactor vessel with a comminuted feed containing mainly proteinaceous material, such as abattoir waste, together with smaller quantities of fruit waste, preferably papayas or an enzymic derivative thereof, and a small quantity of a slowly soluble alkaline material such as a calcarious mineral.

Preferably the feed comprises from 75 to 95 wt % proteinaceous material, for example about 85 wt %.

Preferably the feed comprises from 2.5 to 15 wt % fruit waste, for example about 10 wt %.

Preferably the feed comprises about 2.5 to 10 wt % of a slowly soluble alkaline material, for example about 50 wt %.

Suitably this triple feed mixture is then stirred and lightly oxygenated with air at ambient temperature. As the temperature of the mulch rises the aeration rate is progressively increased.

This gradually increasing temperature reaction condition is preferably maintained until the mulch reaches 70° C. and the microbial culture is pasteurised. Then the aeration rate is preferably further increased. Temperatures of more than 80° C. may then be achieved.

After a period in excess of 1 hour above 80° C. the reaction can be assumed to be complete, and the digestate will be pasteurised. The aeration can then be stopped and the digestate discharged from the reactor.

Suitably the mixture is allowed to remain in the reactor for a period of between 1 and 10 days, for example between 3 and 6 days.

Preferably, throughout the above reaction process a pH of between 5 and 8 is maintained. This may be conveniently achieved via the presence in the reactor of slowly soluble calcararious mineral fines.

It is believed to be important that, although the reaction may approach oxygen starvation it is preferably not allowed to cross the threshold between the desired aerobic state and the undesirable anaerobic condition.

Temperatures above 95° C. are preferably avoided as these can have adverse effects on reactor seals etc.

The nature of the digestate produced by the method of the present invention will now be defined.

Suitably the digestate produced has the consistency and appearance of a soup, containing approximately 20% solids. Preferably, because the digestion process is thorough, virtually all solid materials are present in sub-micron size ranges, or are in the form of a gel.

Within the digestate are suitably contained protein fragments, oligomeric protein, short chain poly peptides, enzymes, modified proteins, proteins with active oxidised chain end groups, and amino acids.

Our research is continuing on the identification and characterisation of specific entities within the digestate produced.

It is an advantageous preferred feature of the digestate produced that the average protein polymer chain length is significantly reduced in comparison to the protein polymers present in conventional aerobic or anaerobic digestates.

These shorter chain length protein-derived species have numerous research, developmental and commercial potentials in the fields of human and animal nutrition, pharmaceuticals and agronomic nutrification.

By basing the feed stock to the digester on different proteinaceous raw materials, different short chain lengths protein-derived entities can be produced.

Our research on the application of this co-enzymically accelerated microbial endenogenous organics process is continuing.

The present invention will next be exemplified in terms of typical ranges of ingredients that can be digested via the process of co-enzymically accelerated microbial enzymic organic digestion:

Typical charges to a 1500 litre aerobic digester are normally within the following preferred ranges:

Proteinaceous abattoir wastes 750 to 950 Kg preferably 850 Kg

Fruit waste eg papaya 25 to 150 Kg preferably 100 Kg

Ground magnesium limestone 25 to 100 Kg preferably 50 Kg

Such a typical charge to the aerobic digester would be made without additional water being added. We have found that the maceration of such a typical charge produces a pumpable slurry.

Such a typical charge would not normally need the addition of steam in the aerobic digester. The accelerated co-enzymic reaction is self initiating, based on microbes attendant with the charge, and proceeds without the provision of addition or auxiliary heating.

Such a typical charge would gradually generate heat as the microbial reaction progresses.

A temperature in the region of 75° C. to 95° C. would normally be achieved from such a typical charge, over a digester dwell time of between three and six days.

The resulting digestate liquor, from such a typical charge, would, after between three and six days, be fully pasteurised and almost completely digested.

The digestate liquor, from such a typical charge would contain:

Amino acids 20 to 40% by weight.

Oligomeric proteins 30 to 60% by weight.

Modified end group proteins 20 to 40% by weight.

Unchanged proteins 5 to 10% by weight.

Other organic materials 5 to 20% by weight.

Such digestate liquors, from typical digester charges, can be used as organic nutrients in:

Animal feeds.

Organic fertiliser.

Organic foliar feeds.

Organic composts and growing mediums.

Such end usages can be served by either the intermediate product, namely the un-modified digestate liquor, or by separated fractions from this liquor, or by dried versions of this liquor.

Taken together the above process and products produced constitute a significant techno-commercial improvement in the manufacture of proteinaceous digestates.

Any feature of any aspect of the present invention may be combined with any other feature, where appropriate.

1. A method of aiding the digestion of a proteinaceous material, the method comprising adding to a sample of proteinaceous raw material a composition comprising fruit, fruit waste or an enzyme derived therefrom.

2. A method according to claim 1 wherein the composition comprising fruit, fruit waste or an enzyme derived therefrom is derived from the papaya family of fruits.

3. A method according to claim 2 wherein the composition comprising fruit, fruit waste or an enzyme derived therefrom comprises papease.

4. A method according to claim 1 wherein the proteinaceous raw material comprises abattoir waste.

5. A method of producing a digestate liquor, the method comprising:
(a) adding to an aerobic digestor a composition comprising a proteinaceous material and a composition comprising fruit, fruit waste or an enzyme derived therefrom; and
(b) allowing the resultant mixture to remain in the reactor for a period sufficient for digestion of the proteinaceous material to occur.

6. A method according to claim 5 wherein in step (a) a slowly soluble alkaline material is added to the reactor.

7. A method according to claim 6 wherein the material added to the reactor in step (a) comprises 75 to 95 wt % proteinaceous material, 2.5 to 15 wt % fruit, fruit waste or an enzyme derived therefrom and from 2.5 to 10 wt % of a slowly soluble alkaline material.

8. A method according to claim 5 wherein in step (b) the mixture is allowed to remain in the reactor for a period of from 3 to 6 days.

9. A method according to claim 5 wherein during step (b) the mixture is maintained at a pH of from 5 to 8.

10. A digestate liquor obtained by a method as claimed in claim 5.

11. (canceled)

12. (canceled)

13. (canceled)

14. A method according to claim 2 wherein the proteinaceous raw material comprises abattoir waste.

15. A method according to claim 3 wherein the proteinaceous raw material comprises abattoir waste.

16. A method according to claim 6 wherein in step (b) the mixture is allowed to remain in the reactor for a period of from 3 to 6 days.

17. A method according to claim 7 wherein in step (b) the mixture is allowed to remain in the reactor for a period of from 3 to 6 days.

18. A method according to claim 6 wherein during step (b) the mixture is maintained at a pH of from 5 to 8.

19. A method according to claim 7 wherein during step (b) the mixture is maintained at a pH of from 5 to 8.

20. A method according to claim 8 wherein during step (b) the mixture is maintained at a pH of from 5 to 8.
